(19) United States
(12) Patent Application Publication
(10) Pub. No.: US 2011/0123651 A1
(43) Pub. Date: May 26, 2011

(54) DIETARY SUPPLEMENT DRINK FOR DELIVERY OF RESVERATROL AND OTHER POLYPHENOLS

(76) Inventors: Thomas E. Mower, Springville, UT (US); Charles Jeffery Brady, Springville, UT (US)

(21) Appl. No.: 12/784,385

(22) Filed: May 20, 2010

Related U.S. Application Data
(60) Provisional application No. 61/180,026, filed on May 20, 2009.

(51) Int. Cl. A61K 31/05; A61K 31/353; A61K 36/53; A61K 36/185; A61K 36/736; A61K 36/45; A61K 31/522; A61K 36/752; A61P 7/02; A61P 35/00; A61P 29/00; A61P 3/10; B82Y 5/00

(52) U.S. Cl. 424/732; 514/733; 514/456; 424/745; 424/776; 424/735; 514/263.31; 424/736; 977/773; 977/904

(57) ABSTRACT

A nutritional beverage or drink is provided having resveratrol mixed with water, suspended in a liquid suspension system. One or more of quercetin, catechins, antioxidants, energy agents, sweeteners, preservatives, pH balancing ingredients, and coloring agents may also be included. A process is provided for preparing a dietary supplement drink in a liquid suspension, comprising mixing water in a suspension system with resveratrol. The process may also include adding one or more of quercetin, catechins, antioxidants, energy agents, sweeteners, preservatives, acidulants, and coloring agents. The nutritional beverage or drink may also be modified by using pure water that has nanosized water clusters therein, as well as mixing into the liquid suspension nanoparticles of resveratrol and/or one or more of the above ingredients to enhance the bioavailability of the ingredients. The compositions may be used to treat diseases and bodily malfunctions.

### CROSS-REFERENCE TO RELATED APPLICATIONS

This application claims the benefit of U.S. Provisional Application No. 61/180,026, filed May 20, 2009, which is hereby incorporated by reference herein in its entirety, including but not limited to those portions that specifically appear hereinafter, the incorporation by reference being made with the following exception: In the event that any portion of the above-referenced provisional application is inconsistent with this application, this application supercedes the above-referenced provisional application.

### STATEMENT REGARDING FEDERALLY SPONSORED RESEARCH OR DEVELOPMENT

Not applicable.

This invention relates generally to dietary supplements containing resveratrol. More particularly, the present invention relates to a dietary supplement drink or beverage for the delivery of resveratrol and other polyphenols and nutritional ingredients, and in illustrative embodiments further comprises a suspension agent for maintaining resveratrol in suspension.

Resveratrol is a naturally occurring substance generated by plants, such as grapes, varieties of knotweed, and dark red to purple berries, in response to environmental stress or pathogenic attack. In grapes it is present only in the skin of the fruit. Resveratrol exists in both the cis and trans isomeric forms, as well as in its glucoside, piceid. In wines and knotweed, the trans form and piceid are by far the most abundant.

Resveratrol has been found to have remarkable anti-aging properties and life extension capabilities in several ways, including decreasing mitochondrial damage associated with aging and disease and increasing mitochondrial biogenesis. In addition, resveratrol has been shown to provide cardioprotective effects, lowering lipid concentrations, inhibiting atherosclerosis, vaso-relaxing activity, inhibition of the aggregation of platelets and LDL oxidation, and antioxidant and anti-carcinogenic properties.

With respect to cardioprotective effects, resveratrol may be responsible for the cardiovascular benefits associated with moderate wine consumption and the “French paradox,” a very low mortality rate due to coronary heart disease in France despite a high-fat diet and smoking habits.

Concerning cancer prevention, it has been shown that resveratrol has anti-carcinogenic properties in mice. An important article published in Science concerning its anti-cancer characteristics is M. Jang et al., Cancer chemopreventive activity of resveratrol, a natural product derived from grapes, 275 Science 218-220 (1997).

Numerous other studies have been conducted with respect to the beneficial aspects of resveratrol. Resveratrol is currently undergoing human clinical trials with the National Institutes of Health in patients with colon cancer, metabolic syndrome, follicular lymphoma, and Alzheimer's disease. Other studies have shown that, in rodents, resveratrol decreases mitochondrial damage associated with aging and disease (cerebral ischemia, myocardial reperfusion injury) and increases mitochondrial biogenesis.

In animals, resveratrol has cardioprotective effects. Resveratrol lowers lipid concentrations and inhibits atherosclerosis in diabetic mice. Resveratrol has shown vaso-relaxing activity in rat and guinea-pig vessels. In vitro and in vivo, resveratrol has been shown to inhibit LDL oxidation. In vitro, resveratrol has antioxidant properties. In addition, resveratrol has been found to block replication of the influenza virus in cell culture and in animals, according to a study conducted by the Institute of Microbiology in Rome.

Large bioactive agents are difficult to present in a soluble or suspended form in aqueous-based compositions. Because of this tendency for such systems to be insoluble, it is apparent that the body also has difficulty in absorbing such bioactive agents. Thus, it would be advantageous to provide an improved delivery system for such bioactive agents.

An illustrative embodiment of the present invention comprises a nutritional drink having resveratrol mixed with water in a liquid suspension. The drink may further comprise a suspension agent for maintaining the resveratrol in suspension. Quercetin may be mixed with resveratrol and water in the liquid suspension. One or more catechins may also be mixed with resveratrol in the liquid suspension, with or without quercetin. An energy agent may also be included in the liquid suspension. The invention may also include one or more antioxidants, flavoring agents, sweeteners, preservatives, acidulants, coloring agents, or mixtures thereof.

Another illustrative embodiment of the invention comprises a process for preparing a dietary supplement drink in a liquid suspension, the process comprising mixing water with resveratrol and, optionally, a suspension agent. The process may include adding quercetin with resveratrol in the liquid suspension. The process may also include combining resveratrol and a catechin in a liquid suspension nutritional drink with or without quercetin. The process may further include adding one or more of an energy agent, an antioxidant, a flavoring agent, a sweetener, a preservative, an acidulant, and a coloring agent.

The nutritional drink may also be modified by using pure water that has nanosized water clusters therein, as well as mixing into the liquid suspension nanosized or nano-encapsulated particles or micelles of resveratrol and/or one or more of the other specified ingredients to enhance the bioavailability of the ingredients.

### DETAILED DESCRIPTION

Before the present resveratrol-containing compositions and methods are disclosed and described, it is to be understood that this invention is not limited to the particular configurations, process steps, and materials disclosed herein as such configurations, process steps, and materials may vary somewhat. It is also to be understood that the terminology employed herein is used for the purpose of describing particular embodiments only and is not intended to be limiting since the scope of the present invention will be limited only by the appended claims and equivalents thereof.

It must be noted that, as used in this specification and the appended claims, the singular forms “a,” “an,” and “the” include plural referents unless the context clearly dictates otherwise. Thus, for example, reference to a dietary supplement drink containing “a suspension agent” includes a mixture of two or more suspension agents, reference to “an energy agent” includes reference to one or more of such energy agents, and reference to “the antioxidant” includes reference to a mixture of two or more antioxidants.

Unless defined otherwise, all technical and scientific terms used herein have the same meanings as commonly understood by one of ordinary skill in the art to which this invention belongs.

In describing and claiming the present invention, the following terminology will be used in accordance with the definitions set out below.

As used herein, “comprising,” “including,” “containing,” “characterized by,” and grammatical equivalents thereof are inclusive or open-ended terms that do not exclude additional, unrecited elements or method steps.

1. Overview of Dietary Supplement Drinks for the Delivery of Resveratrol Alone or with Other Polyphenols and Other Ingredients

The resveratrol composition of the present invention provides many benefits, including without limitation stimulating activity to extend the life span, anti-aging, longevity, repairing mitochondria damage and regenerating new mitochondria, repairing cellular DNA, regenerating youthful tissues, repairing brain and muscle, memory, energy output, vitality, health regeneration and maintenance, promoting bioavailability using nano-sized molecular ingredients, increasing trans-mucosal absorption, removing waste/protein debris that accumulates and builds up within the cells over a lifetime of metabolic activity, promoting weight loss and increasing stamina, athletic strength and endurance, and activating the SIR2 gene and synergistic genes “of youth” to reactivate youthful regeneration.

### A. Resveratrol Nutritional Drink:

Illustrative compositions of the present invention provide resveratrol in a long-term liquid suspension form that makes the resveratrol and other bioactive agents readily available to the body and absorbed by the body. In one illustrative embodiment, resveratrol is combined with pure water and a suspension agent for maintaining resveratrol in suspension. The term “pure water” refers to water that has been purified by a conventional method such as reverse osmosis, distillation, deionization, carbon filtration, microporous filtration, ultrafiltration, ultraviolet oxidation, electrodialysis, or combinations thereof.

In another illustrative embodiment of the present invention, resveratrol is provided in the trans form or piceid, the forms that are by far the most abundant in wines. In one form of resveratrol used in certain embodiments of the present invention, the compositions of the present invention may have a potency equivalent to about 540 four-ounce glasses of wine. The resveratrol used in one illustrative embodiment of the present invention is in dry powder extract, 99% pure. Typically, the resveratrol may be extracted from the skins of grapes or from knotweed or other berries using various methods, including water alcohol extraction. This powder extract may then be mixed with pure water to form the basic composition.

B. Resveratrol Nutritional Drink with Quercetin:

Another illustrative embodiment is comprised of the combination of resveratrol, quercetin, a suspension agent, and pure water in a liquid suspension for use as a nutritional drink. Quercetin has been found to have a synergistic effect when combined with resveratrol to enhance the life-extending and anti-aging effects of resveratrol by working together as stimulatory agents of the SIRT 1 (silent information regulator two homolog 1) gene and other genes for promoting youth, longevity, regeneration, energy, and vitality, as well as enhancing metabolism for weight loss.

Quercetin is a plant-derived flavonoid that has been used as a nutritional supplement, and has been shown to be effective as an anti-inflammatory and anti-oxidant agent. Quercetin also enhances the vitality of the brain, muscles, and organs. Quercetin works in conjunction with the proteins generated by the SIRT1 or SIR2 genes to provide beneficial results. Quercetin may be obtained from an herbal extract, such as Japanese Sophora bark powder.

C. Resveratrol Nutritional Drink with Catechins:

In another illustrative embodiment, a nutritional drink in the form of a liquid suspension may include resveratrol and one or more catechins, with or without quercetin. Catechins are polyphenols mostly derived from green tea (such as a green tea leaf extract), particularly a catechin called epigallocatechin-3-gallate (EGCG), and are also found in the skins of red grapes with resveratrol. Studies of catechins have shown that this polyphenol has many beneficial effects, including the very important ability to promote genetic stimulation for increased proliferation of beneficial cells and to suppress the action of harmful genes, such as the proliferative gene NUDT6 (nucleoside diphosphate-linked moiety X motif 6) as well as to inhibit mutagenic processes. As mentioned above, resveratrol is the primary SIRT1 gene stimulator to promote anti-aging. By adding catechins with resveratrol in a nutritional drink, greater gene action results as stated above. In addition, quercetin may also be added, which is also known as an effective gene stimulator, particularly for the SIRT1 or SIR2 genes, as mentioned above.

Studies have also shown that catechins provide many additional benefits to the body, including without limitation, the promotion of the following: weight loss, antioxidation of free radicals, protection of brain cells and neurons, healthy blood sugar levels to decrease the effects of diabetes, glucose control, cardiovascular health, healthy prostate and ovaries, stimulation of production of anti-mutagenic enzymes and reduction of damage from LDL cholesterol.

D. Resveratrol Nutritional Drink with Antioxidants:

In another illustrative embodiment, a nutritional drink in a liquid suspension with resveratrol may also include an antioxidant. Including an antioxidant in a nutritional drink may be effective for at least two antioxidizing purposes. First, the antioxidant may act on the composition itself to reduce oxidization and thereby preserve the composition. In addition, the antioxidant protects the resveratrol molecules from environmental and digestive system attack to maintain potency, creates a symbiotic cellular DNA cascading effect and increases bioavailability of the resveratrol molecule for absorption into the blood system and to the cells. In addition, the antioxidant also provides flavor to give an agreeable taste to the composition.

E. Resveratrol Nutritional Drink with an Energy Agent and Other Ingredients:

In another illustrative embodiment, an energy agent may also be provided for increasing metabolic rate and promote weight loss, as well as to stimulate youth and extend longevity, energy and vitality. Further, preservation ingredients may be added for preserving the viability of the composition and a sweetener may be included.

### 2. Ingredients of Dietary Supplement Drink for the Delivery of Resveratrol.

The following illustrative embodiments disclose examples of specific ingredients used in a dietary supplement drink for the delivery of resveratrol with respect to the embodiments discussed above. In one illustrative embodiment, resveratrol is provided in combination with quercetin. A suspension agent to maintain resveratrol and quercetin in suspension indefinitely may comprise a combination of sodium citrate, calcium chloride, and a gellan gum such as KELCOGEL® F. The suspension agent maintains the resveratrol in dispersed suspension to maximize the bioavailability of the useful ingredients. A thickener, such as xanthan gum having a brand name TICAXAM™, may also be utilized to enhance suspension and provide a pleasing sensation in the mouth.

Further, an antioxidant may be included in an embodiment of the present invention for several purposes described above. This antioxidant may include one or more of the following ingredients: rosemary extract, pomegranate juice concentrate, plum juice concentrate, red sour cherry juice concentrate, cranberry juice concentrate, and red raspberry juice concentrate. A flavoring agent, such as mixed berry natural flavor may also be added. In addition, sweeteners may be added, including one or more of the following: agave syrup such as XAGAVE® (BetterBody Foods & Nutrition, LLC, Lindon, Utah), and stevia rebaudoside A (for example, from stevia leaf extract).

In addition, an energy agent may be included in an embodiment of the present invention. This energy agent may include one or more of the following ingredients: cha de bugre leaf extract, guarana seed extract, caffeine, green tea 50% polyphenols, green coffee bean extract, and Citrus aurantium, such as a bitter orange concentrate known as ADVANTRA Z®. In particular, the green tea leaf extract (catechins) has been found to act as a stimulatory agent, in conjunction with resveratrol and quercetin, to promote longevity, energy and vitality, as well as enhancing metabolism for weight loss.

In addition, one or more preservatives, such as rosemary extract known as AQUAROX™, and/or sodium benzoate may be included in an embodiment of the present invention. Further an acidulant may be added to adjust pH, such as malic acid and citric acid. In addition, a color agent such as EXBERRY® coloring foodstuff shade “Bordeaux” (GNT USA, Inc., Redwood City, Calif.) may be included in an embodiment of the present invention.

### 3. An Illustrative Formulation Embodiment

An illustrative formulation of a nutritional beverage or drink according to the present invention comprises the following ingredients in parts by weight:

TABLE 1

| Ingredient | Parts by Weight |
|---|---|
| Pure Water | 412.2845 |
| Xanthan gum | 0.3 |
| Gellan gum | 0.15 |
| Sodium citrate | 0.5 |
| Calcium chloride | 0.3935 |
| Agave syrup | 28.75 |
| Pomegranate juice concentrate (65 Brix) | 22.5 |
| Plum juice concentrate(70 Brix) | 10.0 |
| Red sour cherry juice concentrate (68 Brix) | 2.5 |
| Cranberry juice concentrate (50 Brix) | 1.25 |
| Red raspberry juice concentrate (65 Brix) | 10.0 |
| Cha de bugre extract | 3.0 |
| Guarana seed extract | 0.1 |
| Caffeine | 0.335 |
| Green tea 50% polyphenols | 0.25 |
| Citrus aurantium extract | 0.15 |
| Green coffee bean extract | 0.0375 |
| Quercetin | 0.425 |
| Stevia rebaudoside A | 0.2 |
| Grape skin extract (resveratrol) | 2.15 |
| Malic acid | 0.15 |
| Mixed berry natural flavoring | 0.6 |
| EXBERRY ® shade “Bordeaux” | 3.5 |
| Rosemary extract | 0.175 |
| Sodium benzoate | 0.3 |

4. Nanosizing the Water and/or Ingredients

The nutritional beverage or drink may also be modified by using pure water that has nanosized water clusters therein to enhance permeability of the water clusters through human cell walls. The nutritional beverage or drink may be further modified by mixing into the liquid suspension nano-sized particles of resveratrol and/or one or more of the above ingredients to enhance the bioavailability of the ingredients. As used herein, the term “nanosized” means a particle having at least one dimension between 1 and 10 nanometers or a water cluster having no more than 500 water molecules in a cluster.

### 5. A Mixing Process Embodiment

In one illustrative embodiment, the above ingredients are mixed in the following process. The pure water is measured into a mixing tank and heated to about 70° C. The heated water is mixed with a gellan gum, such as KELCOGEL® F, and sodium citrate dihydrate for 4 to 5 minutes to let the gellan gum hydrate. Then the temperature is reduced to 60° C. and calcium chloride 3.7% is mixed until it is completely dissolved.

Next, calcium chloride 3.7% and a xanthan gum such as TICAXAN™ are pre-mixed and then added to the above mixture, which is then mixed for about five minutes to allow the xanthan gum to disperse. At this point, the thickener and suspension agent have been prepared.

Next, an energy ingredient is added to the mixture as follows. At 50° C. or below, the following ingredients are mixed until completely dissolved: cha de bugre PE 10:1, guarana seed PE (39.0-40.0%), caffeine anhydrous USP, green tea 50% polyphenols, a Citrus aurantium extract such as ADVANTRA Z® PE 10, and green coffee bean PE 50%. In addition, stevia rebaudoside A and quercetin are added.

Then, an antioxidant, also providing flavoring, is added and mixed until completely dissolved, increasing speed if necessary to ensure dispersion. The following ingredients may be included: pomegranate juice concentrate, plum juice concentrate, red sour cherry juice concentrate, cranberry juice concentrate, and red raspberry juice concentrate.

At this point the resveratrol is added in the form of grape skin extract (resveratrol 99%) and is mixed until completely dissolved, increasing speed if necessary to ensure dispersion.

The following ingredients are mixed until completely dissolved: malic acid, mixed berry flavor, a coloring agent, such EXBERRY® shade “Bordeaux,” a rosemary extract such as AQUAROX 10, and a preservative such as sodium benzoate. Then these ingredients are added to the composition.

Finally, the composition may be pasteurized by conventional methods.

The resulting nutritionally fortified beverage or drink may be placed in glass or plastic bottles and stored at room temperatures, preferably refrigerated after opening. A daily portion may be about ½ to 2 ounces (about 14.8 to 59.1 mL) taken 1 to 3 times per day with meals.

### 6. Delivery of Resveratrol Composition

One efficient way of administering resveratrol in humans may be buccal delivery, that is without immediate swallowing, by direct absorption through the inside of the mouth. As an example, when one mg of resveratrol in 50 mL solution was retained in the mouth for one minute before swallowing, 37 ng/ml of free resveratrol was measured in plasma two minutes later. This level of unchanged resveratrol in blood can only be achieved with 250 mg of resveratrol taken in pill form.

### 7. Uses of the Compositions

Any of the compositions of the above embodiments may be used in promoting health and proper body functions and in treating various diseases or bodily malfunctions, including without limitation, treating aging, LDL oxidation, mitochondria, platelet aggregation, vasorelaxation, cancer, oxidization, inflammation, fatty liver, diabetes, and insulin resistance.

In addition to the foregoing embodiments, the present invention may be embodied in other specific forms without departing from its spirit or essential characteristics. The described embodiments are to be considered in all respects only as illustrative and not restrictive. The scope of the invention is, therefore, indicated by the appended claims, rather than by the description or examples. All changes that come within the meaning and range of equivalency of the claims are to be embraced within their scope.

The subject matter claimed is:

1. A dietary supplement drink comprising a suspension of resveratrol in water.

2. The dietary supplement drink of claim 1 wherein the resveratrol is in trans form.

3. The dietary supplement drink of claim 1, further comprising quercetin.

4. The dietary supplement drink of claim 1, further comprising a catechin.

5. The dietary supplement drink of claim 4 wherein the catechin comprises epigallocatechin-3-gallate (EGCG).

6. The dietary supplement drink of claim 1, further comprising one or more of an antioxidant, a suspension agent, a thickener, an energy agent, a sweetener, a preservative, an acidulant, a flavoring agent, and a coloring agent.

7. The dietary supplement drink of claim 6 wherein the antioxidant comprises one or more of rosemary extract, pomegranate juice concentrate, plum juice concentrate, red sour cherry juice concentrate, cranberry juice concentrate, and red raspberry juice concentrate.

8. The dietary supplement drink of claim 6 wherein the suspension agent comprises one or more of sodium citrate, calcium chloride, and gellan gum.

9. The dietary supplement drink of claim 6 wherein the thickener comprises xanthan gum.

10. The dietary supplement drink of claim 6 wherein the energy agent comprises one or more of cha de bugre extract, guarana seed extract, caffeine, green tea polyphenols, Citrus aurantium extract, and green coffee bean extract.

11. The dietary supplement drink of claim 6 wherein the sweetener comprises one or more of agave syrup and stevia rebaudoside A.

12. The dietary supplement drink of claim 6 wherein the preservative comprises one or more of rosemary extract and sodium benzoate.

13. The dietary supplement drink of claim 6 wherein the acidulant comprises one or more of malic acid and citric acid.

14. The dietary supplement drink of claim 1, wherein the resveratrol is nanosized, or the water has nanosized water clusters.

15. The dietary supplement drink of claim 1, wherein the water is pure water.

16. A process for preparing a dietary supplement drink, comprising mixing resveratrol with water.

17. The process of claim 16, further comprising adding one or more of a suspension agent, a thickener, quercetin, a catechin, an energy agent, an antioxidant, a sweetener, a preservative, an acidulate, a coloring agent, and a flavoring agent.

18. The process of claim 17 wherein the suspension agent comprises one or more of sodium citrate, calcium chloride, and gellan gum.

19. The process of claim 17, wherein the thickener comprises xanthan gum.

20. The process of claim 17, wherein the catechin comprises epigallocatechin-3-gallate (EGCG).

21. The process of claim 17, wherein the energy agent comprises one or more of cha de bugre extract, guarana seed extract, caffeine, green tea polyphenols, Citrus aurantium extract, and green coffee bean extract.

22. The process of claim 17, wherein the antioxidant comprises one or more of rosemary extract, pomegranate juice concentrate, plum juice concentrate, red sour cherry juice concentrate, cranberry juice concentrate, and red raspberry juice concentrate.

23. The process of claim 17, wherein the sweetener comprises one or more of agave syrup and stevia rebaudoside A.

24. The process of claim 17, wherein the preservative comprises one or more of rosemary extract and sodium benzoate.

25. The process of claim 17, wherein the acidulant comprises one or more of malic acid and citric acid.

26. The process of claim 17, wherein the resveratrol is nanosized, or the water has nanosized water clusters.

27. A dietary supplement drink comprising resveratrol, quercetin, a catechin, and water.
